(19) United States
(12) Patent Application Publication
(10) Pub. No.: US 2005/0159478 A1
(43) Pub. Date: Jul. 21, 2005

(54) Compositions containing d-tocopherol compound polybasic acid partial esters

(76) Inventor: Jeffrey C. Hunsicker, Naperville, IL (US)

Correspondence Address: COGNIS CORPORATION;PATENT DEPARTMENT, 300 BROOKSIDE AVENUE, AMBLER, PA, 19002, US

(21) Appl. No.: 11/017,162

(22) Filed: Dec. 20, 2004

Related U.S. Application Data
(60) Provisional application No. 60/532,120, filed on Dec. 23, 2003.

(51) Int. Cl. A61K 31/355

(52) U.S. Cl. 514/458

(57) ABSTRACT

Compositions comprising a salt of a d-tocopherol compound polybasic acid partial ester of the general formula (I):
[(R—O^{t}(O)C)_{z}-Aₓ-(C(O)O⁻)_{y}]ₙ[Mⁿ⁺]_{y} (I)
wherein each R represents a dextrorotatory tocopherol compound moiety and O^{t} represents the 6-hydroxyl oxygen atom of the dextrorotatory tocopherol compound moiety, A represents a polyvalent hydrocarbon group having from 1 to 44 carbon atoms which can be linear, branched, cyclic or polycyclic, aliphatic or aromatic, saturated or unsaturated and substituted or unsubstituted, x represents 0 or 1, y and z each independently represent a number of from 1 to 4 wherein the sum of y and z equals a number of from 2 to 6, n represents an integer of from 1 to 6 and M represents a metal ion, with the proviso that where x equals zero both y and z each equal 1; and wherein the composition contains an amount of one or more l-tocopherol compounds which is less than 50% by weight of the total tocopherol compound content in the composition.

### CROSS-REFERENCE TO RELATED APPLICATIONS

This application claims priority, under 35 U.S.C. §119(e), of U.S. Provisional Patent Application No. 60/532,120, filed on Dec. 23, 2003, the entire contents of which are hereby incorporated by reference.

### BACKGROUND OF THE INVENTION

Concerns over maintaining healthy lifestyles continue to grow, and accordingly, vitamin and antioxidant use and intake also continue to rise. As more evidence of the potential benefits associated with the use and intake of vitamins and antioxidants continues to be generated, demand for such substances increases, as does the demand for various forms thereof. Many naturally derived antioxidants and vitamins are normally delivered as oily substances or viscous liquids for encapsulation. However, many potential applications for increased, beneficial consumption and use of such vitamins and antioxidants make solid, free-flowing, and/or powdery formulations more desirable.

For example, some tocopherol compounds such as free tocopherol and tocopheryl acetate, which are oily liquids that exhibit vitamin E activity, can be mixed with carriers and other additives to be made into solids. Other ester forms of tocopherol, such as tocopheryl succinate, are solid. Unfortunately, solid forms of tocopherol such as tocopheryl succinate still do not adequately meet all of the applicational demands necessitated by the various forms of desired vitamin consumption, including, for example, compaction for tableting. Tocopherol compound salts, and in particular, salts of dibasic acid hemiesters of tocopherol, provide tocopherol compounds that exhibit some improved formulation properties over simple tocopherol esters. One specific example of such a salt is the calcium salt of tocopherol succinate. Known calcium salts of tocopherol succinate are synthetic, and thus, racemic mixtures of the d- and l-optical isomers.

The properties of dl-tocopherol calcium succinate, with respect to processing unit dosages thereof for animal consumption, such as tablets, are adequate, but improvements would be desirable. The thermal properties of these synthetic, racemic tocopherol salt products are somewhat undesirable in that the heat generated by tableting equipment can cause softening or even melting of the salt which in turn impedes the ability of the tableting equipment to produce quality tablets.

Accordingly, alternatives for vitamins and antioxidants in solid form with advantageous processing properties are desirable. There is a need in the art for such alternative forms of vitamins and antioxidants.

### SUMMARY OF THE INVENTION

The present invention relates, in general, to compositions comprising salts of d-tocopherol compound polybasic acid partial esters. As used herein, the term polybasic refers to any compound having two or more carboxylic acid functionalities and thus includes both dibasic acids and higher functionality polybasic acids. Also, as used herein, the term partial ester refers to any polybasic acid having one or more esterified carboxylic acid groups and one or more unesterified carboxylic acid groups.

More particularly, preferred embodiments of the present invention relate to divalent metal salts of d-tocopherol dibasic acid hemiesters. It has been surprisingly found that salts of d-tocopherol compound polybasic acid partial esters can be produced which exhibit significantly improved softening points. The higher softening points exhibited by the salts of d-tocopherol compound polybasic acid partial esters according to the invention allow for improved processing, such as in tableting operations. Surprisingly, in comparison to synthetic, dl-tocopherol calcium succinate, d-tocopherol calcium succinate exhibits a significantly improved, i.e., increased, softening point. Moreover, the increased softening points exhibited by the salts in accordance with the present invention provide significant improvements in tableting which leads to significant savings in production.

The present invention includes a composition comprising a salt of a d-tocopherol compound polybasic acid partial ester of the general formula (I):
[(R—O^{t}(O)C)_{z}-Aₓ-(C(O)O⁻)_{y}]ₙ[Mⁿ⁺]_{y} (I)
wherein each R represents a dextrorotatory tocopherol compound moiety and O^{t} represents the 6-hydroxyl oxygen atom of the dextrorotatory tocopherol compound moiety, A represents a polyvalent hydrocarbon group having from 1 to 44 carbon atoms which can be linear, branched, cyclic or polycyclic, aliphatic or aromatic, saturated or unsaturated and substituted or unsubstituted, x represents 0 or 1, y and z each independently represent a number of from 1 to 4 wherein the sum of y and z equals a number of from 2 to 6, n represents an integer of from 1 to 6 and M represents a metal ion, with the proviso that where x equals zero both y and z each equal 1; and wherein the composition contains an amount of one or more l-tocopherol compounds which is less than 50% by weight of the total tocopherol compound content in the composition. Preferred metal ions, M, include divalent metals. More preferred metal ions, M, are the alkaline earth metals. In certain preferred embodiments of the present invention, the metal ion, M, comprises a metal ion selected from the group consisting of calcium, magnesium and zinc ions.

In various preferred embodiments of the present invention, the d-tocopherol compound polybasic acid partial ester salt comprises a d-tocopherol compound dibasic acid hemiester salt. In the most preferred embodiments of the present invention, the d-tocopherol compound dibasic acid hemiester salt comprises d-α-tocopherol calcium succinate.

A particularly preferred embodiment of the present invention includes a composition comprising a salt of a d-α-tocopherol compound dibasic acid hemiester of the general formula (I):
[(R—O^{t}(O)C)-A-(C(O)O⁻)]₂ [M²⁺] (I)
wherein R represents a dextrorotatory α-tocopherol compound moiety and O^{t} represents the 6-hydroxyl oxygen atom of the dextrorotatory tocopherol compound moiety, A represents a linear, divalent hydrocarbon group having from 2 to 4 carbon atoms which can be saturated or unsaturated, and M represents a metal ion selected from the group consisting of calcium, magnesium and zinc; and wherein the composition contains an amount of one or more l-tocopherol compounds which is less than 5% by weight of the total tocopherol compound content in the composition.

### DETAILED DESCRIPTION OF THE INVENTION

The present invention includes compositions comprising a salt of a d-tocopherol compound polybasic acid partial ester of the general formula (I):
[(R—O^{t}(O)C)_{z}-Aₓ-(C(O)O⁻)_{y}]ₙ[Mⁿ⁺]_{y} (I).
R represents a dextrorotatory tocopherol compound moiety and O^{t} represents the 6-hydroxyl oxygen atom of the dextrorotatory tocopherol compound moiety. As used herein, the term “tocopherol compound” refers to the broad class of compounds that can be characterized as derivatives of 6-chromanol having an isoprenoid side chain, of which many are known to exhibit vitamin E activity. These compounds include, for example, the alpha (α-), beta (β-), gamma (γ-) and delta (δ-) homologues of tocopherol, as well as unsaturated derivatives, such as, tocomonoenols, tocodienols and tocotrienols.

In certain preferred embodiments of the present invention, the tocopherol compound is α-tocopherol. In certain preferred embodiments of the present invention, the tocopherol compound is γ-tocopherol. In some preferred embodiments of the present invention, the composition comprises a mixture of salts of two or more d-tocopherol compound polybasic acid partial esters of the general formula (I), wherein one of the salts is based upon α-tocopherol and another of the salts is based upon γ-tocopherol. In some preferred embodiments of the present invention, the composition comprises a mixture of salts of d-tocopherol compound polybasic acid partial esters of the general formula (I), wherein the composition comprises salts based upon α-tocopherol, β-tocopherol, γ-tocopherol and δ-tocopherol.

In formula (I), A represents a polyvalent hydrocarbon group having from 1 to 44 carbon atoms, preferably 2 to 18 carbon atoms, more preferably 2 to 12 carbon atoms and most preferably 2 to 4 carbon atoms, which can be linear, branched, cyclic or polycyclic, aliphatic or aromatic, saturated or unsaturated and substituted or unsubstituted. In certain preferred embodiments of the present invention, A represents a divalent or trivalent hydrocarbon group. In more preferred embodiments, A represents a divalent hydrocarbon group. Certain preferred polyvalent hydrocarbon groups can have from 2 to 18 carbon atoms and are linear, and may be saturated or unsaturated. Polyvalent hydrocarbon groups suitable as A in formula (I) may also include one or more heteroatoms and/or bear one or more substituents such as hydroxyl groups, amino groups, carboxylate groups, and thiol groups. In particularly preferred embodiments according to the present invention, A represents a divalent, linear, saturated hydrocarbon group having from 2 to 4 carbon atoms, and most preferably 2 carbon atoms.

In formula (I), M represents a metal ion having a charge, n. The charge, n, can be a number of from 1 to 6. Accordingly, M can represent any metal. In certain preferred embodiments of the present invention, M represents a divalent metal wherein n equals 2. In certain preferred embodiments of the present invention wherein M represents a divalent metal, the divalent metal is an alkaline earth metal. Particularly preferred divalent metals include calcium, magnesium and zinc, with calcium being most preferred.

In formula (I), x can be 0 or 1. In instances where x represents 0, both y and z are equal to 1. Where x equals 1, y and z each independently represent a number of from 1 to 4, and the sum of y+z equals a number of from 2 to 6. In other words, the total number of ester linkages, represented by y, and the total number of free carboxylate groups, represented by z, will be equal to a number of from 2 to 6, when x is equal to 1. In preferred embodiments of the present invention, x equals 1. In certain more preferred embodiments of the present invention, x equals 1 and both y and z represent numbers of from 1 to 2. In certain other preferred embodiments of the present invention, x, y and z each represent 1.

Compositions in accordance with the claimed invention contain an amount of one or more l-tocopherol compounds which is less than 50% by weight of the total tocopherol compound content in the composition. In preferred embodiments of the present invention, the compositions contain an amount of one or more l-tocopherol compounds which is less than 40% by weight of the total tocopherol compound content in the composition. In certain preferred embodiments of the present invention, the compositions contain an amount of one or more l-tocopherol compounds which is less than 30% by weight of the total tocopherol compound content in the composition. In certain more preferred embodiments of the present invention, the compositions contain an amount of one or more l-tocopherol compounds which is less than 25% by weight of the total tocopherol compound content in the composition. In other more preferred embodiments of the present invention, the compositions contain an amount of one or more l-tocopherol compounds which is less than 20% by weight of the total tocopherol compound content in the composition. In even more preferred embodiments of the present invention, the compositions contain an amount of one or more 1-tocopherol compounds which is less than 15% by weight of the total tocopherol compound content in the composition, more preferably less than 10% by weight of the total tocopherol compound content in the composition, more preferably less than 5% by weight of the total tocopherol compound content in the composition, and even more preferably less than 1% by weight of the total tocopherol compound content in the composition. In the most preferred embodiments of the present invention, the compositions contain an amount of one or more l-tocopherol compounds which is below 0.1% by weight of the total tocopherol compound content in the composition.

Salts of d-tocopherol compound polybasic acid partial esters of the general formula (I) in accordance with the present invention can be prepared by reacting a suitable metal reagent with a d-tocopherol compound polybasic acid partial ester. For example, calcium chloride can be reacted with d-α-tocopherol succinate in the presence of ammonia, in accordance with the general process described in U.S. Pat. No. 2,407,725, the entire contents of which are hereby incorporated herein by reference. Alternatively, d-α-tocopherol succinate can be reacted with lithium hydroxide to form the lithium salt of the tocopherol succinate, followed by salt exchange with a metal compound, in accordance with the general process described in U.S. Pat. No. 3,432,525, the entire contents of which are hereby incorporated herein by reference. Salts of d-tocopherol compound polybasic acid partial esters of the general formula (I) in accordance with the present invention can also be prepared by reacting a d-tocopherol compound polybasic acid partial ester, such as d-α-tocopherol succinate, with a metal acetate, such as calcium acetate.

Tocopherol polybasic acid partial ester starting materials which are useful in the processes according to the present invention include reaction products of tocopherol compounds and polybasic acids and/or their anhydrides. Suitable tocopherol compounds are as described above in reference to formula (I). Any carboxylic acid having two or more carboxylic acid groups can be employed.

In preferred embodiments of the present invention, the d-tocopherol compound polybasic acid partial ester starting material comprises a d-tocopherol compound dibasic acid hemiester. Accordingly, suitable d-tocopherol compound polybasic acid partial esters for use in the processes according to the present invention can be prepared by esterifying a d-tocopherol compound of the formula and a polybasic acid of the formula, Aₓ-[C(O)OH]_{q}, or anhydrides thereof; wherein A and x are as defined above in reference to formula (I) and q represents a number of from 2 to 6, preferably 2 to 4, more preferably 2 or 3 and most preferably 2. Preparative esterification procedures suitable for esterifying a d-tocopherol compound and a polybasic acid are known in the art and include any known preparative method for esterifying an alcohol and an acid, such as direct esterification and transesterification using suitable catalysts. For example, an alcohol and a dibasic acid can be reacted in the presence of an acidic catalyst to produce the ester product thereof. A suitable molar ratio of d-tocopherol compound to dibasic acid for the preparation of the dibasic acid hemiester is about 1:1. Where a polybasic acid having more than two acid groups is employed, the molar ratio of tocopherol compound to acid can be selected to achieve the desired number of free carboxylate groups. For example, citric acid having three acid groups can be reacted with a d-tocopherol compound in a molar ratio of d-tocopherol compound to dibasic acid of 1:1 to result in a polybasic acid partial ester having two free carboxylate groups. The ratio can be changed to 2:1 to result in a di-tocopherol polybasic acid partial ester having a single free carboxylate group.

Certain preferred d-α-tocopherol dibasic acid hemiesters which can be reacted with metal reagents to prepare salts and compositions according to various preferred embodiments of the present invention can be obtained commercially from various sources such as Cognis Corporation, available as Covitol® 1210 natural d-α-tocopherol succinic acid, but may also be prepared by reacting one or more tocopherol compounds and a dibasic component selected from dibasic acids, dibasic acid anhydrides, and dibasic acid halides. A preferred route for preparing tocopherol succinic acid for use in the present invention is the direct esterification of d-α-tocopherol with succinic anhydride.

The present invention will now be illustrated in more detail by reference to the following specific, non-limiting examples.

### EXAMPLE 1

Thermal and Rheological Behaviour Analysis of Various Samples of Tocopherol Calcium Succinate:

Differential Scanning Calorimetry:

DSC measurements are conducted with a Perkin Elmer Pyris 1 Differential Scanning Calorimeter. Experiments are conducted between 40° C. and 250° C. at heating/cooling rates of 5.0 and 10.0° C./min. During the experiments, the samples are enclosed in an air tight aluminum pan. The aluminum pan is in a nitrogen purge at 30 psi.

ThermoGravimetric Analysis:

TGA measurements are conducted with a Perkin Elmer TGA 7 Thermogravimetric Analyzer. Experiments are conducted from 30° C. to 250° C. using a heating rate of 10.0° C./min. During the experiments, the sample chamber is purged by nitrogen at a pressure of 30 psi.

Rheology:

Rheology measurements are conducted with TA Instruments Rheolyst AR-1000N rheometer. Experiments are conducted with the parallel plate geometry in a nitrogen atmosphere. The experiments use an oscillation stress of 1.0 Pa at a frequency of 1.0 Hz. The temperature range of the experiments is 40 to 210° C. at a heating rate of 5.0° C./min. Per analogy to rigid sphere or polymers, for TCS samples below the melting point, change in the pseudo G′ and G″ moduli″ is used as an indication of the softening of the sample. The cross-over point corresponds to the point where the ratio of the storage/loss modulus or tan (δ) is equal to one. The temperature at which tan (δ) is equal to one is considered as the softening point for the solid TCS samples. In addition, the second derivative of the loss modulus vs temperature was calculated. A maximum or minimum in the second derivative indicates a curvature or inflexion in the G″ vs T. This can also be taken as indicator of a change in the state of the samples (softening or melting depending on the temperature).

Comparative Results:

Softening points of these tocopherol succinate samples were identified using oscillatory rheology measurements. The results are presented in Table 2 and FIGS. 6 to 9. The temperature at which tan(δ) is equal to one, i.e., cross-over point (or the point where the samples are less rigid) is considered as their softening point. We call the moduli “pseudo loss and storage moduli”, since below the melting point, the powder cannot in reality be viscoelastic.

TABLE 1
Comparison of thermal and rheological behaviour

| | | | Rheology |
|---|---|---|---|
| | | | (oscillatory) @ |
| | DSC | | Tan(δ) = 1 |
| | (heating rate 5° C./min) | | heating |
| | | Enthalpy | Rate(5° C./min |
| | Onset (° C.) | (J/g) | Temperature (° C.) |
| d-α-tocopherol succinic | 74.5 | 80.00 | 74 |
| acid, Covitol ® 1210 | | | |
| dl-tocopherol calcium | 154 | 0.27 | 147 |
| succinate | | | |
| | 176.9 | 0.81 | |
| | 185.8 | 1.50 | |
| d-α-tocopherol calcium | 159.5 | 0.19 | 151 |
| succinate | | | |
| | 179.3 | 1.00 | |
| | 185.4 | 3.90 | |
| d-α-tocopherol calcium | 159.7 | 0.19 | 153 |
| succinate | | | |
| | 179.3 | 0.73 | |
| | 186.5 | 3.92 | |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A composition comprising a salt of a d-tocopherol compound polybasic acid partial ester of the general formula (I):
[(R—O^{t}(O)C)_{z}-Aₓ-(C(O)O⁻)_{y}]ₙ[Mⁿ⁺]_{y} (I)
wherein each R represents a dextrorotatory tocopherol compound moiety and O^{t} represents the 6-hydroxyl oxygen atom of the dextrorotatory tocopherol compound moiety, A represents a polyvalent hydrocarbon group having from 1 to 44 carbon atoms which can be linear, branched, cyclic or polycyclic, aliphatic or aromatic, saturated or unsaturated and substituted or unsubstituted, x represents 0 or 1, y and z each independently represent a number of from 1 to 4 wherein the sum of y and z equals a number of from 2 to 6, n represents an integer of from 1 to 6 and M represents a metal ion, with the proviso that where x equals zero both y and z each equal 1; and wherein the composition contains an amount of one or more 1-tocopherol compounds which is less than 50% by weight of the total tocopherol compound content in the composition.

2. The composition according to claim 1, wherein at least one R represents a d-α-tocopherol moiety.

3. The composition according to claim 1, wherein at least one R represents a d-γ-tocopherol moiety.

4. The composition according to claim 1, wherein x represents 1 and A represents a linear, divalent hydrocarbon group having from 2 to 4 carbon atoms which may be saturated or unsaturated.

5. The composition according to claim 2, wherein x represents 1 and A represents a linear, divalent hydrocarbon group having from 2 to 4 carbon atoms which may be saturated or unsaturated.

6. The composition according to claim 1, wherein y and z each represent 1.

7. The composition according to claim 4, wherein y and z each represent 1.

8. The composition according to claim 5, wherein y and z each represent 1.

9. The composition according to claim 1, wherein n equals 2.

10. The composition according to claim 2, wherein n equals 2.

11. The composition according to claim 4, wherein n equals 2.

12. The composition according to claim 6, wherein n equals 2.

13. The composition according to claim 1, wherein M represents an alkaline earth metal ion.

14. The composition according to claim 1, wherein M represents a divalent transition metal ion.

15. The composition according to claim 1, wherein M represents a metal ion selected from the group consisting of calcium, magnesium and zinc ions.

16. The composition according to claim 2, wherein M represents a metal ion selected from the group consisting of calcium, magnesium and zinc ions.

17. The composition according to claim 4, wherein M represents a metal ion selected from the group consisting of calcium, magnesium and zinc ions.

18. The composition according to claim 6, wherein M represents a metal ion selected from the group consisting of calcium, magnesium and zinc ions.

19. The composition according to claim 1, wherein the composition contains an amount of one or more l-tocopherol compounds which is less than 40% by weight of the total tocopherol compound content in the composition.

20. The composition according to claim 1, wherein the composition contains an amount of one or more l-tocopherol compounds which is less than 25% by weight of the total tocopherol compound content in the composition.

21. The composition according to claim 1, wherein the composition contains an amount of one or more l-tocopherol compounds which is less than 10% by weight of the total tocopherol compound content in the composition.

22. The composition according to claim 1, wherein the composition contains an amount of one or more l-tocopherol compounds which is less than 5% by weight of the total tocopherol compound content in the composition.

23. A composition comprising a salt of a d-α-tocopherol compound dibasic acid hemiester of the general formula (I):
[(R—O^{t}(O)C)-A-(C(O)O⁻)]₂[M²⁺] (I)
wherein R represents a dextrorotatory α-tocopherol compound moiety and O^{t} represents the 6-hydroxyl oxygen atom of the dextrorotatory tocopherol compound moiety, A represents a linear, divalent hydrocarbon group having from 2 to 4 carbon atoms which can be saturated or unsaturated, and M represents a metal ion selected from the group consisting of calcium, magnesium and zinc; and wherein the composition contains an amount of one or more 1-tocopherol compounds which is less than 5% by weight of the total tocopherol compound content in the composition.
